(19) United States
(12) Patent Application Publication
(10) Pub. No.: US 2026/0294756 A1
(43) Pub. Date: Oct. 1, 2026

(54) Organic Natural Deodorant containing Saccharomyces cerevisiae Yeast in Sake Kasu with Glutathione and method of manufacturing

(71) Applicant: Nga Hoang Nguyen, Jersey city, NJ (US)

(72) Inventor: Nga Hoang Nguyen, Jersey city, NJ (US)

(21) Appl. No.: 19/094,833

(22) Filed: Mar. 29, 2025

(51) Int. Cl. A61K 8/27; A61K 8/22; A61K 8/34; A61K 8/64; A61K 8/67; A61K 8/92; A61K 8/9789; A61K 8/99

(52) U.S. Cl. CPC A61K 8/27; A61K 8/22; A61K 8/342; A61K 8/64; A61K 8/676; A61K 8/678; A61K 8/922; A61K 8/927; A61K 8/9789; A61K 8/99

(57) ABSTRACT

This invention presents a natural deodorant comprising Vitamin C, Vitamin E, aloe vera, jojoba oil, zinc ricinoleate, magnesium hydroxide, arrowroot powder, glutathione, probiotics, spirulina, Centella Asiatica, sake yeast, and a blend of nine different essential oils, designed to neutralize odor while nourishing the skin. The composition is clinically tested, and nonallergic to all types of skin, it delivers long-lasting freshness for up to 48 hours, suitable for sensitive skin, reduces hyperpigmentation, and leaves no stains or greasy residue. The product is made of 100% organic and natural ingredients, is free from synthetic fragrances, baking soda, GMOs, alcohol, parabens, and aluminum-based compounds.

### BACKGROUND OF THE INVENTION

### 1. Field of the Invention

The present invention is related to a natural deodorant composition comprising vitamin C, vitamin E, Aloe vera, Shea butter, Zinc Ricinoleate, Kokum butter, glutathione, Arrowroot Powder, probiotics to control odor, Spirulina Platenesis extract, Centella Asiatica (CICA), jojoba oil, sake yeast, blend of plurality of natural essential oils and environment-friendly methods of making such composition. This deodorant offers long, fresh, and attractive up to 48 hours, safe for all skin types.

### 2. Description of the Related Art

Conventional deodorants contain harmful ingredients like Baking Soda, parabens, alcohol, synthetic fragrances, aluminum-based compounds, synthetic fragrances, and other chemicals which disrupt skin health and cause irritation. Aluminum-based compounds are proven by research to cause breast cancer. Parabens, and contribute to hyperpigmentation and Underarm Darkening. Aluminum-based compounds like aluminum chlorohydrate and aluminum zirconium antiperspirants are commonly used, which interfere with the body's natural detox process by blocking sweat glands, these traps toxins inside body instead of allowing them to be expelled, leading to long-term health risks. These synthetic alcohol-based fragrances may contain phthalates which can cause allergic reactions, itching, redness, and irritation. There is a need for natural, effective, safe and skin-friendly alternatives. Present invention introduces a non-toxic deodorant without any side effects.

### BRIEF SUMMARY OF THE INVENTION

The present invention presents a natural deodorant comprising ingredients like naturally sourced minerals, probiotics, plant-based ingredients, and blend of essential oils to neutralize odor without using toxic artificial chemicals. The formulation contains Zinc Ricinoleate, Ascorbic acid, glutathione, probiotics, Spirulina Platenesis extract, Centella Asiatica, coconut oil, refined shea butter, aloe vera, jojoba oil, and sake yeast, among other natural components. The composition of the present deodorant controls odor with health benefits to the skin, offering long-lasting freshness without leaving stains or greasy residue.

The present deodorant was evaluated through 48 hours long on skin semi-occlusive patch test, conducted on 30 subjects of various age groups and results indicate that the formulation is well-tolerated, even by individuals with sensitive skin, and can be safely used without causing irritation or any adverse reactions. The formula is approved by dermatologists (HRIPT patch test) and suitable for all skin type, especially sensitive skin due to CICA and Aloe vera properties.

### DETAILED DESCRIPTION OF THE INVENTION

This deodorant composition of the present invention is formulated to provide long-lasting odor protection, causing no skin allergy or irritation and be gentle with sensitive skin. The required ingredients are divided into four batches which first need to be mixed separately then mixed to form a final compound.

The formulation process begins with first batch “A”, comprising beeswax (cera alba, 0.01-30%), Cetearyl Alcohol (0.01-10%), Zinc Ricinoleate (0.01-10%), coconut oil (cocos nucifera, 0.01-30%), Cetyl Alcohol (0.01-10%), Stearyl Alcohol (0.01-10%), refined shea butter (butyrospermum parkii 0.01-30%), and Kokum butter (garcinia indica seed butter, 0.01-30%). These ingredients form the base composition and provide viscous texture, ensuring stability and thermal resistance. Adding beeswax, cetearyl alcohol, and kokum butter into coconut oil increases the melting point 92° F. (instead of normal coconut always melting at 67° F.), these ingredients offer moisturizing properties. Beeswax, shea butter, and Kokum butter contribute to the product's smooth texture and help form a protective barrier over the skin, preventing moisture loss. The inclusion of Cetearyl Alcohol, Cetyl Alcohol, and Stearyl Alcohol enhances consistency and gives the deodorant its smooth glide on skin during application.

Ingredients in second batch B consist of ingredients such as Magnesium Hydroxide (0.01-30%), arrowroot powder (maranta arundinacea root powder, 0.01-30%), each contributing to odor control and moisture absorption. CICA or Centella asiatica extract (0.01-5%), Ascorbic acid (Vitamin C, 0.01-5%), Lactobacillus Fermentum (0.01 -5%), Glutathione (0.01-5%), and sake yeast (Saccharomyces Ferment, 0.01-5%). Magnesium Hydroxide neutralizes odor-causing bacteria while maintaining a pH balance that is gentle on the skin. Arrowroot Powder absorbs excess moisture, preventing discomfort caused by perspiration. The CICA substance are beneficial in calming skin and make the product suitable for all skin types, Vitamin C and glutathione works for armpit appearance improvement. Vitamin C can create a slight acidic environment for probiotics working and killing bacteria while simultaneously brightening the skin and reducing hyperpigmentation. Lactobacillus Fermentum, a probiotic, helps maintain a healthy balance of skin flora, curbing odor production. Glutathione, a powerful antioxidant, further contributes to brightening the underarm area and neutralizing free radicals. Sake Yeast, rich in enzymes and nutrients, helps nourish the skin while providing additional antimicrobial benefits.

Third batch C is blend of Spirulina Platenesis extract in Jojoba oil (Spirulina (1-20%) in Jojoba oil, 0.01-5%) with Coco Caprylate (0.01-10%), Fison EcoSil (Hydrogenated Ethylhexyl Olivate and Hydrogenated olive oil unsaponifiables, 0.01-10%), and Xanthan gum (0.01-3%). Spirulina Platenesis extract is packed with antioxidants, vitamins, minerals, it nourishes and revitalizes the skin while supporting the deodorant's natural odor-neutralizing action. Jojoba oil provides hydration without clogging pores, It also has antibacterial and antioxidant benefits. Coco Caprylate and Fison EcoSil act as a lightweight emollient that ensures smooth application and improves texture, hydration, and smoothness of the deodorant while enhancing the deodorant's texture and providing a soft, smooth and silky feel. Xanthan Gum is a natural thickener which improves the product's consistency.

Fourth batch “D” includes Aloe vera oil (Aloe Barbadensis oil, 0.01-10%), Tocopherol (0.01-5%), Grape seed oil (Citrus Paradisi fruit extract, 0.01-5%), Phytocide Elderberry preservative (Sambucus Nigra fruit extract, 0.1-5%) and a customized blend of nine natural essential oils (0.01-2%). Aloe vera oil is a natural moisture that moist and hydrates the skin and kills bacteria, tocopherol acts as an antioxidant, protecting the skin from environmental damage. Grape seed oil offers additional antioxidant protection and aids in maintaining the skin's elasticity, reducing inflammation. The blend of essential oils provides a sophisticated fragrance, the oils can be customized to achieve the desired scent.

The production process follows strict temperature control to preserve the integrity of the natural ingredients between 130° F.-180° F. A double boiler is used to melt the ingredients in the batch “A” to ensure that waxes dissolve completely without degrading any heat-sensitive compounds. Batch “C” and “D” are then mixed into batch “A”, followed by batch “B”, ensuring uniform distribution. The resulting mixture is poured into containers and left to harden and stabilize for 1-4 hours.

The deodorant formulation is lab-certified and dermatologically tested through semi-occlusive patch testing conducted on a cohort of 30 subjects of various age groups. The test revealed no instances of erythema or edema, classifying the product as “Not Irritating”. The results demonstrate the product's suitability for sensitive skin, ensuring it can be safely used without causing irritation or any adverse reactions.

What is claimed is:

1. A method of making a natural deodorant formulation, where ingredients are mixed in the separate four batches, comprising:
First batch of ingredients comprising: beeswax, Cetearyl Alcohol, Zinc Ricinoleate, coconut oil, Cetyl alcohol, Stearyl alcohol, refined shea butter, and kokum butter;
Second batch of blending ingredients comprising: Spirulina Platenesis extract in jojoba oil, coco caprylate, Fison EcoSil, and Xanthan gum;
Third batch of blending ingredients comprising: aloe vera oil, tocopherol, grape seed oil, Phytocide Elderberry preservative and a blend of essential oils;
Fourth batch of blending ingredients comprising: magnesium hydroxide, arrowroot powder, Centella Asiatica extract, ascorbic acid, lactobacillus Fermentum, glutathione, and sake yeast.

2. The mixing process of the batches as claimed in claim 1 is further completed into four steps comprising:
The first step is to blend and heat the said first batch until the blend liquefies and turns homogeneous and the temperature of the said blend is up to 180° F.;
The second step is to mix the second batch with said first batch when the until the blend is homogeneous;
The third step is to mix the third batch with the blend of the first and second batches until the blend is homogeneous;
The fourth step is to mix the fourth batch with the blend of the first, second, and third batches until the blend is homogeneous and mixture temperature is between 140° F. to 155° F. and ready to be solidified.

3. The second step as claimed in claim 2, is to be carried out when the temperature of the blend of the first batch reduces to below 160° F.

4. The third step as claimed in claim 2, is to be carried out when the temperature of the blend of the first and the second batches reduces to below 160° F.

5. A natural deodorant formulation comprises:
a) Beeswax in an amount from 0.01% to 30% by weight of said formulation;
b) Cetearyl Alcohol in an amount from 0.01% to 10% by weight of said formulation;
c) Zinc Ricinoleate in an amount from 0.01% to 10% by weight of said formulation;
d) Coconut Oil in an amount from 0.01% to 30% by weight of said formulation;
e) Cetyl Alcohol in an amount from 0.01% to 10% by weight of said formulation;
f) Stearyl Alcohol in an amount from 0.01% to 10% by weight of said formulation;
g) Refined Shea Butter in an amount from 0.01% to 30% by weight of said formulation;
h) Kokum Butter in an amount from 0.01% to 30% by weight of said formulation;
i) Magnesium Hydroxide in an amount from 0.01% to 30% by weight of said formulation;
j) Arrowroot Powder in an amount from 0.01% to 30% by weight of said formulation;
k) Centella Asiatica Extract in an amount from 0.01% to 5% by weight of said formulation;
l) Ascorbic Acid (Vitamin C) in an amount from 0.01% to 5% by weight of said formulation;
m) Lactobacillus Fermentum in an amount from 0.01% to 5% by weight of said formulation;
n) Glutathione in an amount from 0.01% to 5% by weight of said formulation;
o) Sake Yeast in an amount from 0.01% to 5% by weight of said formulation;
p) Spirulina Platenesis extract in Jojoba Oil in an amount from 0.01% to 5% by weight of said formulation;
q) Coco Caprylate in an amount from 0.01% to 10% by weight of said formulation;
r) Fison EcoSil in an amount from 0.01% to 10% by weight of said formulation;
s) Xanthan Gum in an amount from 0.01% to 3% by weight of said formulation;
t) Aloe Vera Oil in an amount from 0.01% to 10% by weight of said formulation;
u) Tocopherol in an amount from 0.01% to 5% by weight of said formulation;
v) Grape Seed Oil in an amount from 0.01% to 5% by weight of said formulation;
w) Essential Oil Blend in an amount from 0.01% to 2% by weight of said formulation;
x) Phytocide Elderberry preservative in an amount from 0.1 to 5% by weight of said formulation.
